(19) United States
(12) Patent Application Publication
(10) Pub. No.: US 2017/0319939 A1
(43) Pub. Date: Nov. 9, 2017

(54) SPORTS APPARATUS FOR PROVIDING INFORMATION

(71) Applicant: Aboense Oy, Turku (FI)

(72) Inventors: Juho Aaltonen, Turku (FI); Hugo Leppäsyrjä, Espoo (FI)

(21) Appl. No.: 15/492,372

(22) Filed: Apr. 20, 2017

(30) Foreign Application Priority Data
May 6, 2016 (FI) 20165390

(51) Int. Cl. A63B 71/06; A61B 5/024; A63B 24/00; A61B 5/00

(52) U.S. Cl. CPC A63B 71/0622; A61B 5/02438; A61B 5/7445; A61B 5/02416; A63B 24/0062; A63B 2207/02; A61B 2562/0219; A63B 2220/40; A63B 2220/803; A63B 2220/836; A63B 2230/062; A63B 2071/0658

(57) ABSTRACT

A device for providing information to a user, the device including means for measuring heart rate of the user, a sensor for detecting an orientation of the device, a light source for emitting a light beam from the device to render the information in a surface outside of the device; and at least one actuator for adjusting relative direction of the light beam in respect to the orientation of the device.

### FIELD

Present disclosure is related to a device for presenting information to a user.

### BACKGROUND

There are various devices offered for athletes to monitor bio signals (such as heart rate) and/or speed of the athlete during exercise or competition. One example of such a device is a watch having a global positioning system (GPS) sensor and means for measuring heart rate. Typically such devices have a user interface to present a parameter such as heart rate or speed to the user. A problem with visual user interfaces is that they require the user to change pace of the training while taking a look on the user interface (of the wrist watch for example). One solution to overcome the problem is to have vibrator or similar in the device to provide alert via vibration to the user, when for example the heart rate is higher than a pre-set level or when the user runs too slow or too fast. This solution has limitations of not providing detailed information related to the training for the user.

### SUMMARY

A device according to the present disclosure is a device for providing information to a user, the device comprising

means for measuring heart rate of the user;

a sensor for detecting orientation of the device;

a processor for creating information from the measured heart rate;

a light source for emitting a light beam from the device to render the information on a surface outside the device; and

at least a first actuator for adjusting relative direction of the light beam with respect to the orientation of the device.

A system according to the present disclosure is a system for providing information to a user, the system comprising;

means for measuring a parameter related to the user;

means for using the measured parameters to create information;

a device for rendering the information to the user; the device comprising

a sensor for detecting an orientation of the device;

a light source for emitting a light beam from the device to render the information on a surface outside the device; and

at least a first actuator for adjusting relative direction of the light beam with respect to the orientation of the device.

### BRIEF DESCRIPTION OF THE DRAWING

FIG. 1A is a schematic illustration of a setup according to an embodiment seen from a side,

FIG. 1B is a schematic illustration of the setup of FIG. 1A, seen from above,

FIG. 2A is an illustration of a device according to an embodiment seen from a side,

FIG. 2B is an illustration of the device of FIG. 2A seen from the front,

FIG. 3A is an illustration of mechanics of a device according to an embodiment seen from above,

FIG. 3B is an illustration of the mechanics of the device of

FIG. 3A seen from the side,

FIG. 4 is a schematic illustration of a setup according to an embodiment seen from a side,

FIG. 5A is an example of correlation between distance for rendering the information and heart rate,

FIG. 5B is an example of correlation between the angle of light from the device with respect to the ground and heart rate,

FIG. 6 is a schematic illustration of some functional modules of the device,

FIG. 7a is a schematic illustration of a setup according to an embodiment seen from a above,

FIG. 7b is a schematic illustration of a setup according to an embodiment seen from a above

FIG. 7c is a schematic illustration of a setup according to an embodiment seen from a side and

FIG. 8 is a schematic illustration of the mechanics of the device according to an embodiment.

### DETAILED DESCRIPTION

A device according to the present disclosure is a device for providing information to a user, the device comprising

means for measuring heart rate of the user;

a sensor for detecting orientation of the device;

a processor for creating information from the measured heart rate;

a light source for emitting a light beam from the device to render the information on a surface outside the device; and

at least a first actuator for adjusting relative direction of the light beam with respect to the orientation of the device,

wherein the information is rendered in the form of a marker on a ground and the distance between the device and the marker is a function of the measured heart rate.

The device can be used to render information such as heart rate of the user or indication of the heart rate of the user for the user, using a beam of light emitting from the device. Rendering of the information refers to illuminating a surface such as the ground with the beam of light. The device has a sensor to detect orientation of the device when the device is used by the user. The orientation information is used to determine where to direct the beam of light from the device with respect to the orientation of the device. The direction of the light can be adjusted with one or more actuators. The device has the advantage that it solves the problem of needing to change pace of training to take a look on user interface in a watch or similar. The device also enables giving detailed information related to training for the user.

According to an embodiment, the sensor is an accelerometer or a gyroscope. The sensor is used to determine the orientation of the device. The accelerometer can be a 3-way accelerometer for detecting orientation of the device with respect to X, Y and Z axis.

According to an embodiment, the first actuator is arranged to rotate at least part of the device to change direction of the light. The rotation of a part of the device changes the direction of the light beam. Further, the rotation of at least a part of the device might be done in order to stabilize the device or to provide rough direction to the light. Further, the rotation can be used to compensate for the up and down movement of the device when attached to a moving user. Further, the rotation can be used to compensate head movements of the user. The rotation can be used to keep the overall orientation of the light source in a pre-determined angle in relation to the ground (i.e. a horizontal plane).

According to an embodiment, the device further comprises

a mirror arranged in a path of the light beam, and

a second actuator connected to the mirror and arranged to rotate the mirror to change direction of the light beam.

The second actuator can be used to rotate one or more mirrors to adjust direction of the beam of light. The second actuator can be used to change orientation of a mirror to which the light beam is directed, to change direction of the light beam. The second actuator can also be used to render text and/or graphics.

According to an embodiment, the light beam is a laser light.

According to an embodiment, the means for measuring the heart rate is a photoplethysmography sensor (PPG).

The information is rendered in the form of a marker on a ground and the distance between the device and the marker is a function of the measured heart rate. According to another embodiment, the device can use heart rate information to determine a distance from the device where the marker should be rendered. Further, the device can use the speed of the user (i.e. device) to determine where the marker should be rendered.

In one embodiment, the marker is further away from user when the heart rate is low or below a set threshold to indicate that user should speed up the training (for example running). As the user sees the marker far away, it is an indication for trying to catch up the marker, thus the heart rate would increase. On the other hand, the marker could be configured to be close to user when the heart rate is higher than the determined target rate. This would indicate to the user to slow down to decrease the heart rate. The marker could be set to be for example to 3-4 meters in front of the user when the heart rate is at the target.

An alternative embodiment is to set target speed for the user (for running or walking or cycling exercise). The rendered marker in the ground would be further away from the user if the speed is too low and close to user if the speed is too high. The marker could be set to be for example to 3-6 meters in front of the user when the speed is at the target.

Alternatively to a marker, the beam of light could be used to render text, numbers and/or graphics on the ground. The text could indicate for example the heart rate or the speed of the user. The rendered graphics could be at a pre-determined distance from the user all the time or the distance could vary depending on the heart rate or the speed of the user as explained above in connection with the marker.

According to an embodiment, the device comprises attachment means for attaching the device in an ear of the user. Alternatively, the device can be attached to the head of the user with a band. Alternatively, the device can be attached to other body parts such as chest of the user.

A system according to the present disclosure is a system for providing information to a user, the system comprising

means for measuring a parameter related to the user;

means for using the measured parameters to create information;

a device for rendering the information to the user; the device comprising

a sensor for detecting an orientation of the device;

a light source for emitting a light beam from the device to render the information on a surface outside the device; and

at least a first actuator for adjusting relative direction of the light beam with respect to the orientation of the device,

wherein the information is rendered in a form of a marker on a ground and the distance between the device and the marker is a function of the measured parameter, and the parameter is one of
a heart rate of the user, measured with a heart rate monitoring device,
a speed of the user, measured with a location sensor.

According to an embodiment, the means for measuring the parameter is a portable computing device. The portable computing device can be for example a smart phone, a phone, a heart rate monitor, a global position system (GPS) device, a speedometer or similar.

The heart rate of the user can be measured with a heart rate monitoring device or with the device, and the speed of the user can be measured with a location sensor or speedometer. The location sensor can be for example a GPS.

According to an embodiment, the portable computing device can be configured to communicate with the device. The portable computing device can be configured to collect measurement data from the device and/or it can be configured to provide information to the device such as audio to be played with the device. Further the portable computing device can be configured to provide measurement information over communication network to server system.

### DETAILED DESCRIPTION OF THE DRAWING

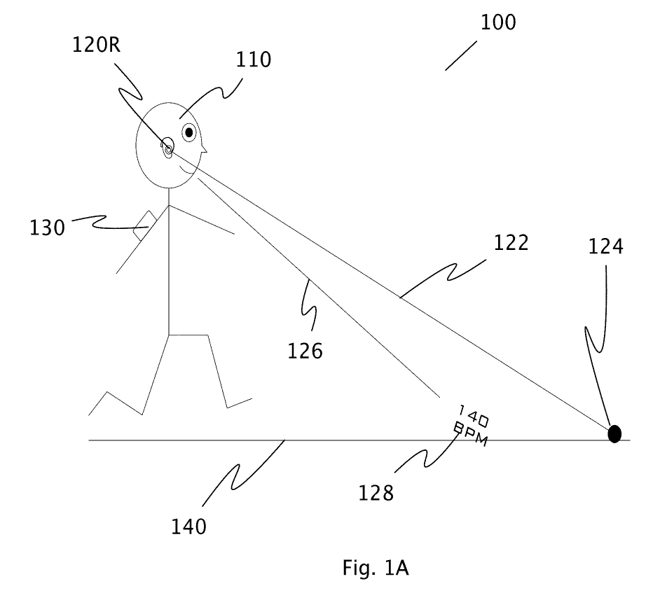

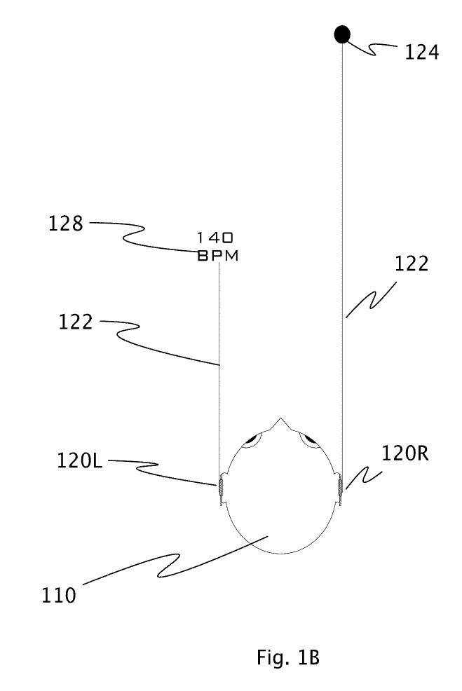

FIG. 1A and FIG. 1B provide an illustration of an environment 100 according to an embodiment. A user 110 with two devices 120R and 120L attached in his ears is illustrated. The device 120L is attached to the left ear and the device 120R is attached to the right ear of the user 110. The devices 120R and 120L have a light source to render information for the user 110. The information is rendered on the ground 140.

The light beam 122 from the device 120R is used to render a dot 124 on the ground 140 and the light beam 126 from the device 120L is used to render a text and numbers on the ground 140. In the present example, text and numbers could indicate for example the heart rate of the user 110.

The user 110 has a portable computing device 130 attached to his arm. The portable computing device 130 can be used to send and receive information to/from the device 120L and/or 120R. A Bluetooth connection can be used for wireless communication. The devices 120R and 120L can be also connected with wires to the portable communication device and/or to each other. The information from the device can be for example the measured heart rate of the user. The information from the portable computing device can be for example audio. The portable computing device 130 can be used to communicate with external services and also to determine location of the user using global position system (GPS) of the portable computing device 130.

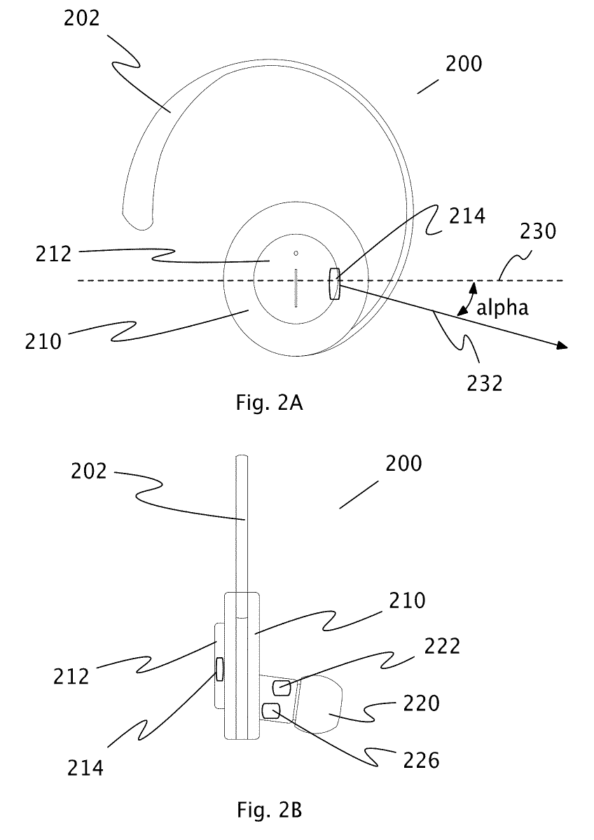

FIG. 2A is a side view illustration of a device 200. FIG. 2B is a front view illustration of the device 200. The device 200 has attachment means 202 to attach the device 200 to an ear of a user. The device 200 has a main body part 210 and beamer part 212. The beamer part 212 has an optically transparent opening 214 for directing light beam 232 from the device 200.

The dotted line in FIG. 2A represents a horizontal line 230 in respect to the ground. The device 200 has an orientation sensor to detect direction of the ground. The orientation sensor is an XYZ-accelerometer sensor of which readings can be used to detect the direction of the ground with respect to the device 200. The beam 232 is directed from the device 200 with angle alpha in relation to the horizontal line 230.

The device has a plug 220 which can be inserted in the ear of the user. The plug is used to provide audio from speaker 226 to the user. The plug has an infrared (IR) transmitter receiver pair 222 to detect the heart rate of the user. The IR pair 222 measures heart rate using photoplethysmogram (PPG).

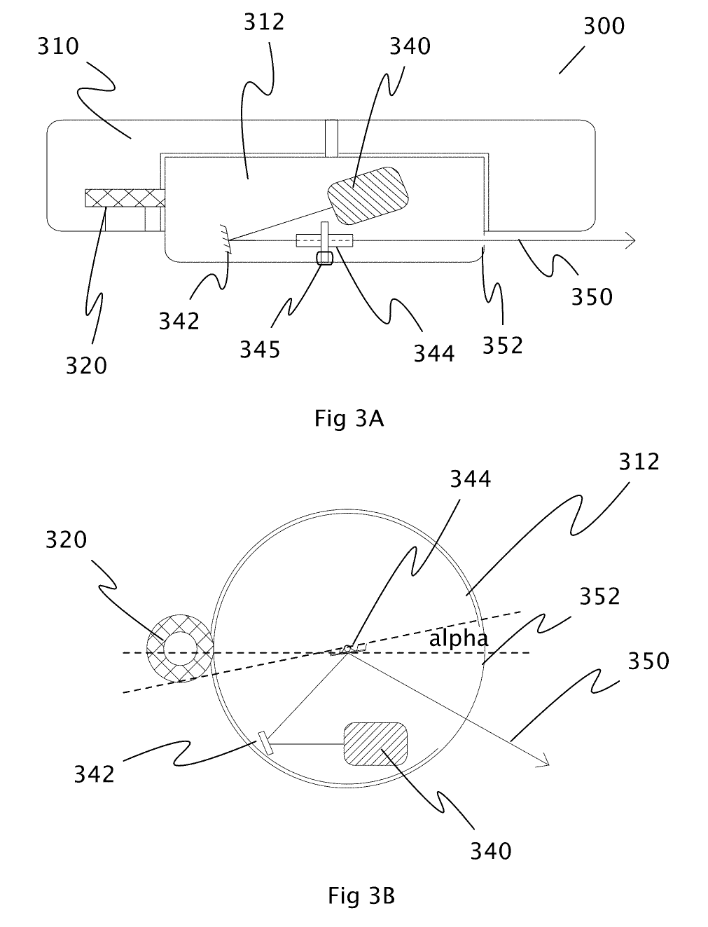

FIG. 3A is an illustration of some of the mechanics of a device 300 seen from top. FIG. 3B. is an illustration of some of the mechanics of the device 300 seen from side.

A stabilizing actuator 320 is connected to a body part 310. The stabilizing actuator 320 is used to rotate the beamer part 312. The rotation causes a change in the angle between a horizontal line and a beam 350. This is used to a) direct the light to an appropriate distance from the user and b) to keep the direction of light with respect to a horizontal line substantially constant to compensate head movements. A rotation angle is controlled by a microcontroller in the device 300.

A light beam 350 is generated by a light source 340. The light beam 350 from the light source is reflected from a first mirror 342 to a second mirror 344. The beam of light 350 goes through an optically transparent opening 352. The first and second mirrors 342 and 344 are actuated with a first actuator and a second actuator 345. The second and first actuators are used to control direction of the light beam 350 to render text, numbers and/or images on the ground at an appropriate distance (as adjusted with the stabilizing actuator). In one embodiment the first mirror 342 is attached to the beamer part and the second mirror 344 is actuated by a second mirror actuator 345 (with a motor or servo). The first actuator can thus be the same that is used for adjusting the relative direction of the light beam, or it can be a separate, mirror actuator.

Alternatively, the stabilizing actuator 320 is used to keep the beamer part 312 at a pre-determined orientation with respect to the ground and the mirror actuators are used to control distance of graphics or dots from the user.

The device 300 can be used to provide a simple indication to the user. As an example, a dot of light is rendered on the ground at distance from the user. The distance between the dot of light and the user is longer when the heart rate is low. The distance between the dot of light and the user is shorter when the heart rate is high. This enables an intuitive way for the user to adjust his/her pace of running or walking or cycling. A target is to get the dot to say at a predetermined distance from the user, not too far (too low heart beat thus not exercising sufficiently) and not too close (too high heart beat). In an embodiment, the distance of the dot from the user is controlled by rotating the beamer part 312 with the stabilizing actuator 320. In case of rendering text or other graphics on the ground, the rotation is used to define a centre area for the rendered text or graphics and the mirror actuators are used to draw the graphics in proximity of the centre area.

Further, in FIG. 3B dashed lines are drawn to illustrate an angle alpha between a horizontal line going through a mid point of the beamer part 312 and a stabilizing actuator 320 and a mirror 344.

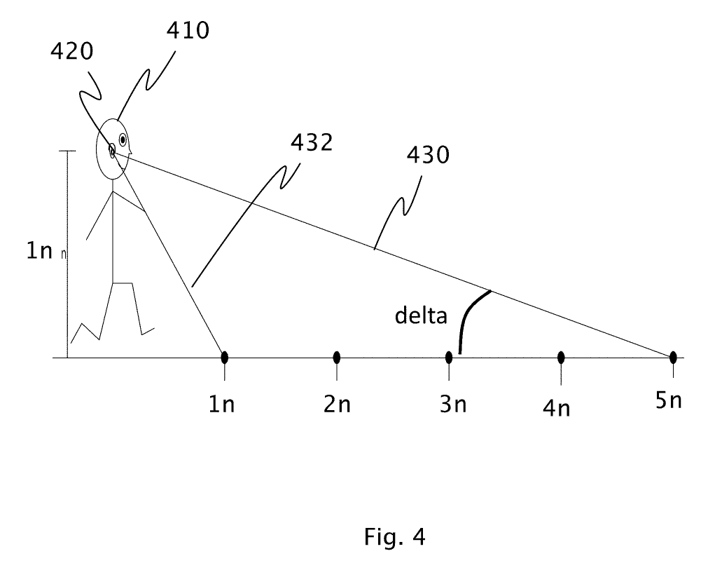

FIG. 4 is an illustration of how the distance of the light dot created by a beam of light 430, 432 on the ground from the user 410 can be adjusted. The user has the device 420 attached to his/her ear at a height 1n from the ground. The light beam 430 creates a dot at a distance 5n and a light beam 432 at distance 1n.

Based on an embodiment, either the stabilizing actuator or the second mirror is used to adjust the distance of the dot from the user by adjusting the angle of light emitting from the device 420 with respect to the device 420. Based on an embodiment, the distance of the dot from the user is a function of the heart rate. If the heart rate is in the desired range the dot is illuminated at a distance between 2n and 3n. If the heart rate is higher than the desired rate, the dot is illuminated at a distance between 1n and 2n. If the heart rate is lower than desired rate, the dot is illuminated at a distance greater than 3n.

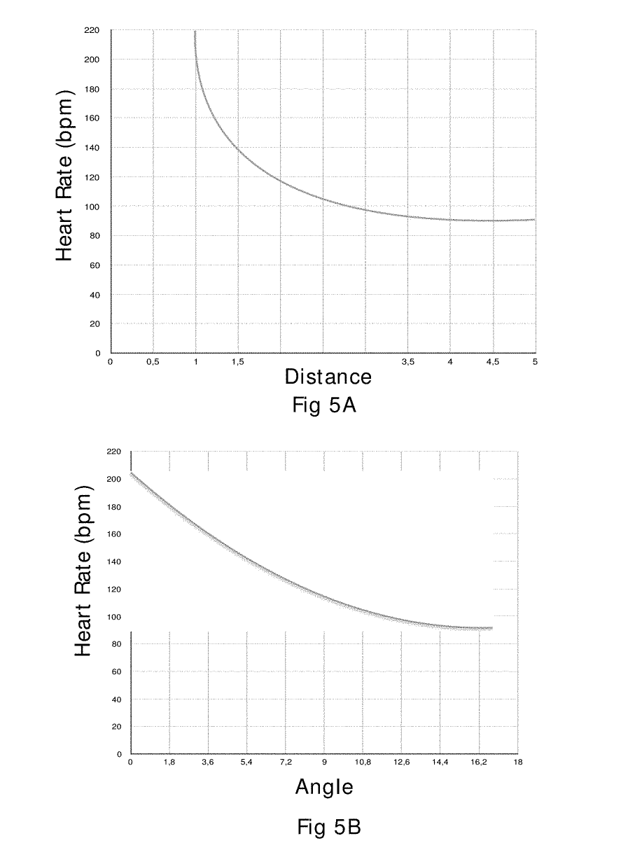

FIG. 5A shows an example correlation curve between the distance of the dot and the heart rate. FIG. 5B shows an example of the angle of the actuator mirror with respect to the horizontal line for the respective heart rate.

One way to calculate the angle between a second mirror 344 and the horizontal line is using equation

α=22,5°−0,5δ

wherein delta is a desired angle between the ground and the beam.

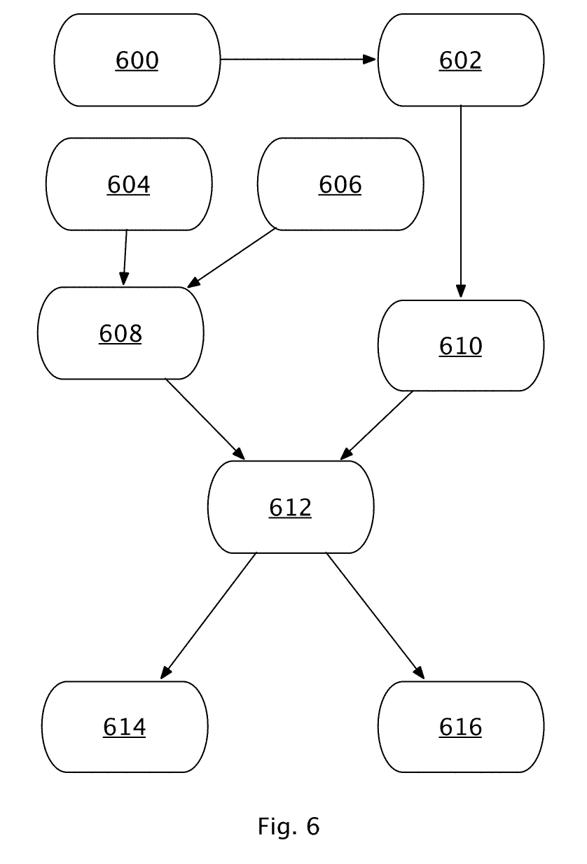

FIG. 6. is an illustration of modules related to the device and some information flows between the modules. A heart rate monitor module 600 provides heart rate related data or information such as beat-to-beat frequency to a heart rate data analyser module 602. The analysed data is used to make control parameters related to directing light from the device in a heart rate function module 610.

An orientation sensor module 604 (such as gyroscope or accelerometer) provides orientation information with relation to the ground. The information from the sensor module 604 is processed with an algorithm from an accelerator processing module 606.

Information from the orientation sensor module 604 is analysed in a movement and orientation analyser module 608 using an algorithm and parameters from the accelerator processing module 606 to determine possible periodic or semi-periodic movements of the user as well as the angle of the device with respect to the ground.

As an example, movements can be predicted with a function taking in consideration running/walking habits of the user. Acceleration of the device can be estimated as function of time, t as:

$$a ( t ) = \frac{h}{2} {( \frac{π v}{s} )}^{2} cos ( \frac{ π v}{s} t )$$

where h is a vertical distance change of the device, when it moves with the head when in use (in practice ear height variation from the ground), v is speed of the user and s is a length of the step. This enables prediction of when and how to tilt mirrors or activate stabilizing actions. In practice maximum acceleration is when a feet hits ground. Accelerations of 5-8 m/ŝ2 were observed when testing the system.

Information from the movement and orientation analyser module 608 and information from the heart rate function module 610 are received with a central processing unit (CPU) module 612. The CPU module 612 provides control signals for stabilizing the actuator motor controlling module 614 to rotate the beamer part of the device. The CPU module 612 provides control signals to the control actuators controlling module 616 moving the first and/or second mirror.

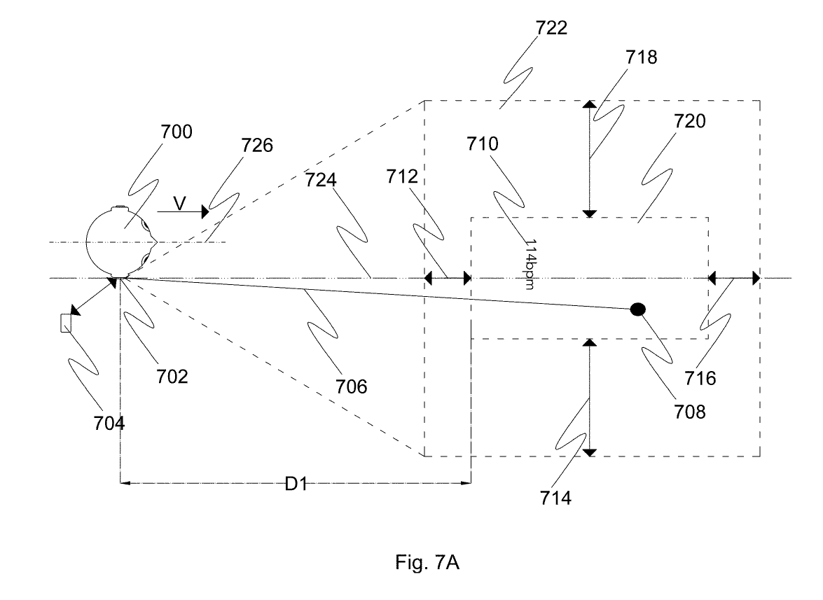

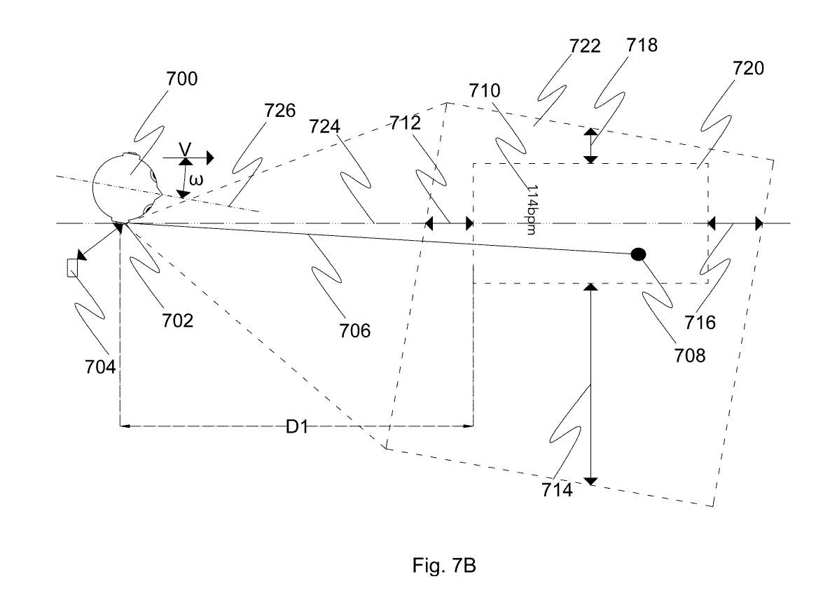

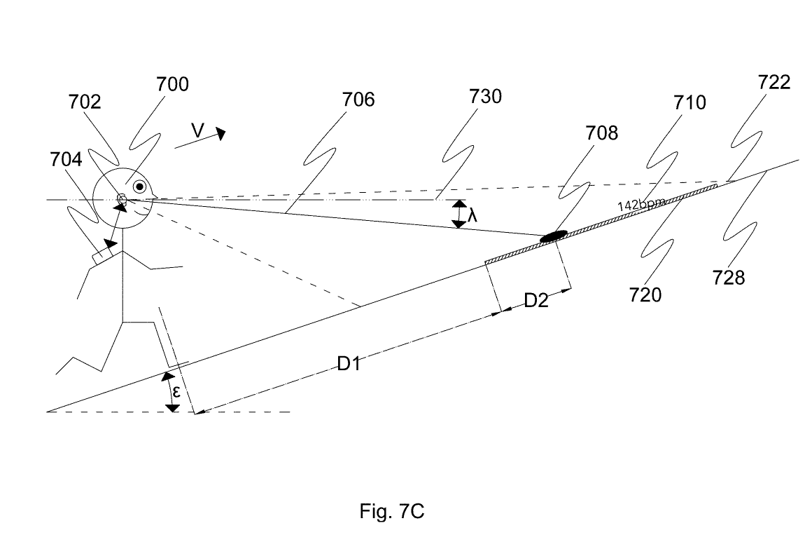

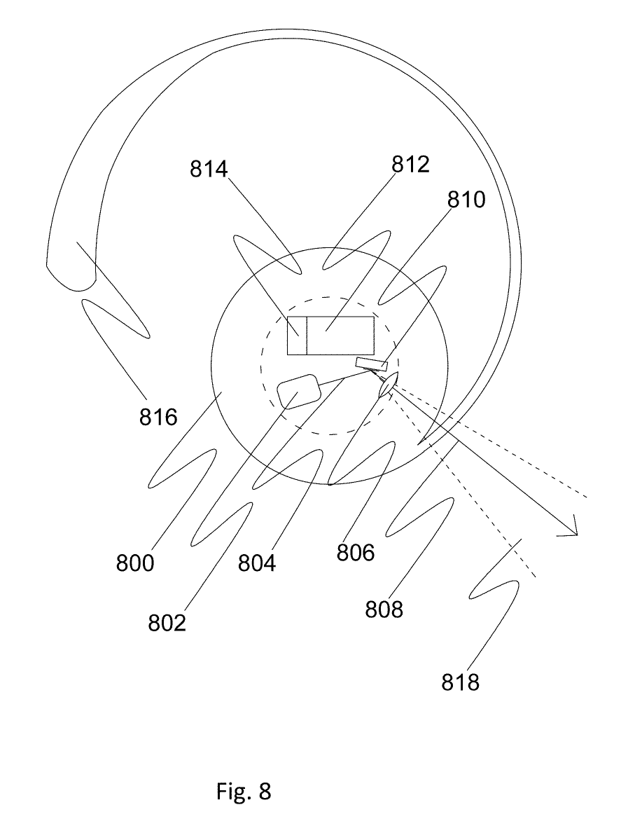

FIG. 7a is an illustration according to an embodiment of present disclosure. A user 700 (seen from top) has a device 702 attached to his head. The device 702 is connected over radio interface to a portable computing device 704. Direction of the head is indicated with line 726. The device is configured to render information within a total rendering area 722 in a ground. Information is rendered in an information rendering area 720 of the total rendering area 722.

The device 700 and/or portable computing device 704 is configured to determine velocity vector V of the user 700. The velocity vector V indicates speed and direction of the speed (in X, Y and Z components). The velocity vector V is determined by measuring velocity and direction of velocity using sensors of the device 702 and/or the portable computing device 704. Example sensors which can be used are an accelerometer or a GPS. The velocity vector can be instantaneous velocity or it can be average of velocities over time. The device 700 and/or portable computing device 704 is configured to calculate for example using Kaman filters (or moving averages) velocity vector V to eliminate random movements and rapid head movements. Alternatively direction of the velocity vector can be determined by environment where the exercise is taken place. For example if runner is in standard 400m oval track the velocity vector can be turned automatically to follow the track of where the user is running. As another example a map can be used to determine if the velocity vector direction has to be changed.

Velocity vector V is used to determine relative location of the information rendering area 720 in respect to user 700 when the user 700 is using the device 702. The information rendering area 720 is in parallel to the velocity vector V. Line 724 illustrates direction of vector V as well as centre line of the information rendering area 720.

A laser beam 706 is used to render a marker 708 and/or parameter info 710 such as text and numbers (heart beat for example) in the ground. The information is rendered in the information rendering area 720. The information rendering area 720 is distance 718 from left side of the total information rendering area 722, distance 714 from right side of the total information rendering area 722, distance 712 from the front and distance 716 from the rear of the total rendering area 722.

The information rendering area 720 is at distance D1 from the user. Information placement within the information rendering area is varied depending on the measured parameter. For example the marker can be further a way from user 700 if the heart beat is low (or speed is slow) and closer to user 700 if the heart beat is high (or speed is high).

FIG. 7B is an illustration of a situation where the head of the user 700 has turned angle of w in respect to velocity vector V and direction of the head. Since the velocity vector V has not changed (user is running in same direction) the information rendering area 720 stays in relation to user 700 in the same relative position i.e. at distance D1 in a direction of the velocity vector V. Since the head has turned the angle w the total information rendering area has moved as indicated in the figure. The information is thus rendered in direction of velocity vector V.

Example: Uphill running (FIG. 7C)

FIG. 7C is an illustration of a user 700 running uphill of a slope having an angle □ in respect to horizontal. The total information rendering area 722 is indicated with dashed line. The information rendering area 720 is illustrated and it is at distance D1 from the user 700. A marker 708 is rendered with a beam of laser 706 originating from the device 702 in the ground 728. The marker 708 is rendered at distance D2 from the edge of the information rendering area 720 i.e. at distance D1+D2 from the user 700. Additional information such as a heart beat 710 can be also rendered with the laser light 706. The device 702 can have an orientation sensor to detect orientation of the device 702 in respect to the horizontal. Horizontal line in respect to device 702 is indicated with a line 730.

When using the system a physiological parameter such as heart beat or velocity is determined using sensor in the device 702, in the portable computing device 704 or other device such as a pulse meter. The parameter determines a distance (D1+D2) from user 700 where the marker 708 is rendered. Distance D1 is user settable value such as 1 meter. The distance D2 can be determined for example by equation:

$$D 2 = \frac{HR}{55 \frac{1}{min \cdot m}}$$

wherein HR is a hearth rate (beats per minute). Value 55 is a user selectable parameter. If the heart beat is 220 beats per minute the equation will give D2 as 4 meters (i.e. the marker is at distance of 1 m+4 m=5 m). If the heart beat is 110 the D2 is 2 meters (i.e. the marker is at distance of 1 m+2 m=3 m).

Velocity vector V is determined by measuring direction of movement and velocity using accelerometer and/or GPS information. The velocity vector V has angle □□ in respect to the horizontal.

Angle □ between horizontal line 730 and the laser beam 706 for rendering marker at distance D1+D2 from the user can be determined for example by equation:

$$λ = arctan ( \frac{h}{D 1 + D 2} ) - ɛ$$

wherein h is height of the device 702 from the ground i.e. for example 160 cm if ear of the user 700 is 160 cm from the ground.

This way the marker can be rendered at the distance D1+D2 from the device independently if user is running on flat surface (□=0) or uphill □>0 or downhill □<0). Needed values for the calculation is the direction of the velocity vector V and height of the device in respect to ground when in use.

FIG. 8 is an illustration of alternative embodiment of the device 800. A laser source 802 emits a light beam 808 via a first actuator (microelectromechanical systems (MEMS) mirror) 810 and a lens system 806. The first actuator (MEMS mirror) 810 is controlled by central processing unit (CPU) 812 to render information with the laser light 808 in an information rendering area 818. The device 800 comprises sensors 814 for detecting orientation and/or accelerometer of the device 800. The sensor information is processed in the CPU 812 to determine velocity vector and orientation to determine area for rendering the information. The device 800 can have radio interface such as Bluetooth to connect to external device such as smart phone or biosensor (such as heart rate measurement sensor) to receive parameter related to user (heart rate or velocity). The device 800 is connected to ear with attachment means 816. A battery can be embedded in the attachment means. CPU uses the parameters to determine content and a relative distance of the content from the device to be rendered.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A device for providing information to a user, the device comprising
means for measuring heart rate of the user;
a sensor for detecting orientation of the device;
a processor for creating information from the measured heart rate;
a light source for emitting a light beam from the device to render the information on a surface outside the device; and
at least a first actuator for adjusting relative direction of the light beam with respect to the orientation of the device,
wherein that the information is rendered in the form of a marker on a ground and the distance between the device and the marker is a function of the measured heart rate.

2. A device according to claim 1, wherein the sensor is an accelerometer or a gyroscope.

3. A device according to claim 1, wherein the first actuator is arranged to rotate at least part of the device to change direction of the light beam.

4. A device according claim 3, further comprising
a mirror arranged in a path of the light beam and
a second actuator connected to the mirror and arranged to rotate the mirror to change direction of the light beam.

5. A device) according to claim 1, wherein the light beam is a laser light.

6. A device according to claim 1, wherein the means for measuring heart rate is a photoplethysmography sensor.

7. A device according to claim 1, wherein the device further comprises attachment means for attaching the device in an ear of the user

8. A system for providing information to a user the system comprising;
means for measuring a parameter related to the user;
means for using the measured parameters to create information;
a device for rendering the information to the user; the device comprising
a sensor for detecting an orientation of the device;
a light source for emitting a light beam from the device to render the information on a surface outside the device; and
at least a first actuator for adjusting relative direction of the light beam with respect to the orientation of the device,
wherein that the information is rendered in a form of a marker on a ground and the distance between the device and the marker is a function of the measured parameter, and the parameter is one of
a heart rate of the user, measured with a heart rate monitoring device,
a speed of the user, measured with a location sensor.

9. A system according to claim 8, wherein the means for measuring the parameter is a portable computing device.

10. A system according to claim 8, wherein the system further comprises means for determining velocity vector of the user.
